(19) United States
(12) Patent Application Publication
(10) Pub. No.: US 2016/0189394 A1
(43) Pub. Date: Jun. 30, 2016

(54) METHOD FOR ITERATIVELY EXTRACTING MOTION PARAMETERS FROM ANGIOGRAPHY IMAGES

(71) Applicant: Huazhong University of Science and Technology, Wuhan (CN)

(72) Inventors: Tianxu ZHANG, Wuhan (CN); Zhenghua HUANG, Wuhan (CN); Lihua DENG, Wuhan (CN); Yaozong ZHANG, Wuhan (CN); Qiong SONG, Wuhan (CN)

(21) Appl. No.: 14/960,461

(22) Filed: Dec. 7, 2015

Related U.S. Application Data
(63) Continuation-in-part of application No. PCT/CN2015/072681, filed on Feb. 10, 2015.

(30) Foreign Application Priority Data
Dec. 30, 2014 (CN) 201410844139.4

(51) Int. Cl. G06T 7/20; G06T 17/00; A61B 6/00; G06T 7/00

(52) U.S. Cl. CPC G06T 7/206; G06T 7/0012; G06T 7/2033; G06T 17/005; A61B 6/504; G06T 2207/30104; G06T 2207/10116; G06T 2207/20056

(57) ABSTRACT

A method for extracting motion parameters from angiography images using a multi-parameter model. The method includes: 1) extracting I vascular structural feature points automatically from a medical image of an angiography image sequence, and auto-tracking the feature points respectively in the angiography image sequence to obtain a tracking sequence of each feature point; 2) performing a discrete Fourier transformation on the tracking sequence of each feature point to obtain a discrete Fourier transformation result; initializing an iterative parameter, and obtaining amplitude range and frequency range of each frequency point of the discrete Fourier transformation result; 3) performing a Fourier transformation on a tracking sequence of each frequency point in the amplitude range and the frequency range thereof to obtain Fourier transformation results; and 4) performing an inverse Fourier transformation on the Fourier transformation results, and obtaining an estimated minimum mean square error of each frequency point.

### CROSS-REFERENCE TO RELATED APPLICATIONS

This application is a continuation-in-part of International Patent Application No. PCT/CN2015/072681 with an international filing date of Feb. 10, 2015, designating the United States, now pending, and further claims priority benefits to Chinese Patent Application No. 201410844139.4 filed Dec. 30, 2014. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND OF THE INVENTION

1. Field of the Invention

The invention relates to a method for iteratively extracting motion parameters from angiography images using a multi-parameter model.

2. Description of the Related Art

Typically, coronary angiography images record not only projection of cardiac movement on a 2D surface, but also 2D movement of coronary arteries on an angiograph surface. Thus, to reconstruct a 3D cardiovascular tree from dynamic 2D angiograms, the cardiac movement and breathing movement must be separated.

A typical method for separation of the cardiac movement and breathing movement includes presetting labels in human organs such as heart and diaphragm, and tracking the labels to obtain a motion curve as approximate breathing movement. The method requires many trials and, thus, is inapplicable for clinical applications.

Another method is to obtain parameters of the breathing movement and cardiac movement by double-armed X-ray angiography. The method is relatively complex, requires harmful contrast agents, and the motion parameters obtained thereby are not accurate enough.

### SUMMARY OF THE INVENTION

In view of the above-mentioned problems, it is an objective of the invention to provide a method for iteratively extracting motion parameters from angiography images using a multi-parameter model, so as to solve technical problems of failing to extract translational movement automatically and separate breathing movement and cardiac movement accurately in prior art.

The invention provides a method for iteratively extracting parameters of multiple movements (including translational movement, breathing movement and cardiac movement) from an X-ray angiography image sequence, selecting a few feature points on coronary arteries, obtaining motion curves thereof by tracking them in an image sequence, optimizing components of the movements under the condition of a minimum mean square error between a reconstructed signal and an original signal at frequency points in frequency domain through a discrete Fourier transformation and an inverse discrete Fourier transformation and using a multi-parameter model, and obtaining optimized 2D motion curves for cardiac movement, tremor movement, breathing movement and translational movement, which is applicable for clinical applications.

To achieve the above objective, according to one embodiment of the present invention, there is provided a method for iteratively extracting motion parameters from angiography images using a multi-parameter model, the method comprising:
(1) extracting/vascular structural feature points automatically from a medical image of an angiography image sequence, and auto-tracking the feature points respectively in the angiography image sequence to obtain a tracking sequence {sᵢ(n), i=1, . . . , I} of each feature point, where n is frame number of the medical image in the angiography image sequence;
(2) performing a discrete Fourier transformation on the tracking sequence {sᵢ(n), i=1, . . . , I} of each feature point in (1) to obtain a discrete Fourier transformation result Sᵢ(k);
(3) initializing an iterative parameter j=0, and obtaining an amplitude range and a frequency range of each frequency point of the discrete Fourier transformation result Sᵢ(k) in (2);
(4) performing a Fourier transformation on a tracking sequence of each frequency point in the amplitude range and the frequency range thereof to obtain Fourier transformation results;
(5) performing an inverse Fourier transformation on the Fourier transformation results in (4), and obtaining an estimated minimum mean square error of each frequency point;
(6) determining whether the estimated minimum mean square error is greater than a predetermined threshold, and proceeding to (7) if yes, otherwise ending the process;
(7) processing spectrums of each frequency point by a multi-parameter iterative optimizing algori^{th}m to obtain (j+1)^{th} iterated time-domain signals;
(8) processing a residual signal by a translation model to obtain a (j+1)^{th} iterated translation signal;
(9) adding the (j+1)^{th} iterated time-domain signals to the (j+1)^{th} iterated translation signal to obtain an (j+1)^{th} iterated estimated mixed signal, and calculating a (j+1)^{th} iterated minimum mean square error; and
(10) determining whether the (j+1)^{th} iterated minimum mean square error is greater than the threshold in (6), and returning to (7) if yes, otherwise ending the process.

In a class of this embodiment, in (1), sᵢ(n) is expressed by the following equation:

sᵢ(n)=L(n)+rᵢ(n)+cᵢ(n)+hᵢ(n)+tᵢ(n),i∈[1, I],

where L(n) represents translational movement, rᵢ(n) represents breathing movement of an i^{th} feature point, cᵢ(n) represents cardiac movement of the i^{th} feature point, hᵢ(n) represents tremor movement of the i^{th} feature point, and tᵢ(h) represents other movements of the i^{th} feature point.

In a class of this embodiment, in (2), Sᵢ(k) is expressed by the following equation:

Sᵢ(k)=L(k)+Rᵢ(k)+Cᵢ(k)+Hᵢ(k),

where k represents a frequency point, and L(k), C(k), R(k) and H(k) represent harmonic coefficients of L(n), c(n), r(n) and h(n) correspondingly and respectively.

In a class of this embodiment, in (5), the estimated minimum mean square error \hat{ε}ᵢ^{j} of the frequency point is expressed by the following equation:

$${\hat{ɛ}}_{i}^{j} = min ( \frac{1}{N} \sqrt{{\sum }_{n} {( {s}_{i} ( n ) - {\hat{s}}_{i}^{j} ( n ) )}^{2}} ) ,$$

where ŝᵢ^{j}(n)=L^{j}(n)+rᵢ^{j}(n)+cᵢ^{j}(n)+hᵢ^{j}(n).

In a class of this embodiment, (7) further comprises:

(7.1) calculating values L^{j}(k_{ic}), Rᵢ^{j}(k_{ic}) and Hᵢ^{j}(k_{ic}) of L^{j}(k), Rᵢ^{j}(k) and Hᵢ^{j}(k) near a frequency point k_{ic} in the frequency range respectively by the following equation while keeping L^{j}(k), Rᵢ^{j}(k) and Hᵢ^{j}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated cardiac signal spectrum by an equation Cᵢ^{j-1}(k_{ic})=Cᵢ⁰(k_{ic})−Rᵢ^{j}(k_{ic})−Hᵢ^{j}(k_{ic}), performing a discrete Fourier transformation thereon in the frequency range, and obtaining an optimized (j+1)^{th} cardiac time-domain signal cᵢ^{j+1}(n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where ω is a frequency point after the discrete Fourier transformation, M represents a window size which is set to 3 normally, and Ŝᵢ^{j}(k)==L^{j}(k)+Rᵢ^{j}(k)+Cᵢ^{j+1}(k)+Hᵢ^{j}(k);

(7.2) calculating values Lⁱ(kᵢₕ), Rᵢ^{j}(kᵢₕ) and Cᵢ^{j+1}(kᵢₕ) of L^{j}(k), Rᵢ^{j}(k) and Cᵢ^{j+1}(k) near a frequency point kᵢₕ in the frequency range respectively by the following equation while keeping L^{j}(k), Rᵢ^{j}(k) and Cᵢ^{j+1}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated high-frequency signal spectrum by an equation Hᵢ^{j+1}(kᵢₕ)=Hᵢ⁰(kᵢₕ)−L^{j}(kᵢₕ)−Rᵢ^{j}(kᵢₕ)−Cᵢ^{j+1}(kᵢₕ), performing a discrete Fourier transformation thereon in the frequency range, and obtaining an optimized (j+1)^{th} high-frequency time-domain signal h^{j+1} (n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where Ŝᵢ^{j}(k)==L^{j}(k)+Rᵢ^{j}(k)+Cᵢ^{j+1}(k)+Hᵢ^{j}(k); and

(7.3) calculating values L^{j}(kᵢᵣ), Hᵢ^{j+1}(kᵢᵣ) and Cᵢ^{j+1}(kᵢᵣ) of L^{j}(k), Hᵢ^{j+1}(k) and Cᵢ^{j+1}(k) near a frequency point kᵢᵣ in the frequency range respectively by the following equation while keeping L^{j}(k), Hᵢ^{j+1}(k) and Cᵢ^{j+1}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated breathing signal spectrum by an equation Rᵢ^{j+1}(kᵢᵣ)=Rᵢ⁰(kᵢᵣ)−L^{j}(kᵢᵣ)−Cᵢ^{j+1}(kᵢᵣ)−Hᵢ^{j+1}(kᵢᵣ), performing a discrete Fourier transformation thereon in the frequency range, and obtaining an optimized (j+1)^{th} breathing time-domain signal rᵢ^{j+1}(n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where Ŝᵢ^{j}(k)==L^{j}(k)+Rᵢ^{j+1}(k)+Cᵢ^{j+1}(k)+Hᵢ^{j+1}(k).

In a class of this embodiment, in (9), the (j+1)^{th} iterated minimum mean square error \hat{ε}ᵢ^{j+1} is expressed by the following equation:

$${\hat{ɛ}}_{i}^{j + 1} = min ( \frac{1}{N} \sqrt{{\sum }_{n} {( {s}_{i} ( n ) - {\hat{s}}_{i}^{j + 1} ( n ) )}^{2}} ) .$$

According to another embodiment of the present invention, there is provided a system for iteratively extracting motion parameters from angiography images using a multi-parameter model, the system comprising:
a first module, operable for extracting I vascular structural feature points automatically from a medical image of an angiography image sequence, and auto-tracking the feature points respectively in the angiography image sequence to obtain a tracking sequence {sᵢ(n), i=1, . . . , I} of each feature point, where n is frame number of the medical image in the angiography image sequence;
a second module, operable for performing a discrete Fourier transformation on the tracking sequence {sᵢ(n), i=1, . . . , I} of each feature point derived by the first module to obtain a discrete Fourier transformation result Sᵢ(k);
a third module, operable for initializing an iterative parameter j=0, and obtaining amplitude range and frequency range of each frequency point of the discrete Fourier transformation result Sᵢ(k) derived by the second module;
a fourth module, operable for performing a Fourier transformation on a tracking sequence of each frequency point in the amplitude range and the frequency range thereof to obtain Fourier transformation results;
a fifth module, operable for performing an inverse Fourier transformation on the Fourier transformation results derived by the fourth module, and obtaining an estimated minimum mean square error of each frequency point;
a sixth module, operable for determining whether the estimated minimum mean square error is greater than a predetermined threshold, and proceeding to a seventh module if yes, otherwise ending the process;
a seventh module, operable for processing spectrums of each frequency point by a multi-parameter iterative optimizing algori^{th}m to obtain (j+1)^{th} iterated time-domain signals;
an eighth module, operable for processing a residual signal by a translation model to obtain a (j+1)^{th} iterated translation signal;
a ninth module, operable for adding the (j+1)^{th} iterated time-domain signals to the (j+1)^{th} iterated translation signal to obtain an (j+1)^{th} iterated estimated mixed signal, and calculating a (j+1)^{th} iterated minimum mean square error; and
a tenth module, operable for determining whether the (j+1)^{th} iterated minimum mean square error is greater than the threshold in the sixth module, and returning to the seventh module if yes, otherwise ending the process.

Advantages of the method for iteratively extracting motion parameters from angiography images using a multi-parameter model according to embodiments of the invention are summarized as follows:

1. with (2), the invention obtains structural feature points automatically for motion curves, avoiding setting labels on surface of the heart;

2. with the optimizing method by multiple iterations in (7), the invention solves problems of failing to extract translational movement automatically and separate other periodic movements (such as breathing movement and cardiac movement); and

3. with the optimizing method by multiple iterations in (7), the invention solves problems of low robustness with inaccurate extracted motion parameters.

### BRIEF DESCRIPTION OF THE DRAWINGS

FIG. 1 is a flow chart of a method for iteratively extracting motion parameters from angiography images using a multi-parameter model of the invention;

FIG. 2 is an original angiography image obtained from an angle;

FIG. 3 illustrates feature points selected from the vascular skeleton in FIG. 2;

FIGS. 4A and 4B are original motion curves of feature points A1-A4 in FIG. 2 in an X direction and a Y direction;

FIGS. 5A and 5B are spectrum components of the fourth feature point in FIG. 2 in the X direction and the Y direction;

FIGS. 6A and 6B are cardiac motion curves of feature points in FIG. 2 in the X direction and the Y direction;

FIGS. 7A and 7B are breathing motion curves of feature points in FIG. 2 in the X direction and the Y direction;

FIGS. 8A and 8B are high-frequency motion curves of feature points in FIG. 2 in the X direction and the Y direction;

FIGS. 9A and 9B illustrate translational motion curves of feature points in FIG. 2 in the X direction and the Y direction compared with those obtained by tracking skeleton points manually;

FIGS. 10A and 10B are residual curves of feature points in FIG. 2 in the X direction and the Y direction;

FIG. 11 is an original angiography image obtained from another angle;

FIG. 12 illustrates feature points selected from the vascular skeleton in FIG. 11;

FIGS. 13A and 13B are original motion curves of feature points A1-A5 in FIG. 11 in an X direction and a Y direction;

FIGS. 14A and 14B are spectrum components of the fourth feature point in FIG. 11 in the X direction and the Y direction;

FIGS. 15A and 15B are cardiac motion curves of feature points in FIG. 11 in the X direction and the Y direction;

FIGS. 16A and 16B are cardiac motion curves of feature points in FIG. 11 in the X direction and the Y direction;

FIGS. 17A and 17B are breathing motion curves of feature points in FIG. 11 in the X direction and the Y direction; and

FIGS. 18A and 18B are residual curves of feature points in FIG. 11 in the X direction and the Y direction.

### DETAILED DESCRIPTION OF THE EMBODIMENTS

For clear understanding of the objectives, features and advantages of the invention, detailed description of the invention will be given below in conjunction with accompanying drawings and specific embodiments. It should be noted that the embodiments are only meant to explain the invention, and not to limit the scope of the invention.

Technical terms of the invention are illustrated as follows.

Minimum mean square error: a minimum value of a mean-squared superimposition of differences between estimated signals and original signals. Multi-parameter model of the invention obtains optimized extracted signals by a method of minimum mean square error performing iterations in time domain and frequency domain to achieve a minimum residual signal in terms of the property of a discrete Fourier transformation.

To solve the problems in prior art, the invention provides a multi-parameter model of structural feature points, selecting a few vascular structural feature points from angiography images, obtaining motion curves thereof by tracking them in an image sequence, optimizing components of the movements under the condition of a minimum mean square error between a reconstructed signal and an original signal at frequency points in frequency domain through a discrete Fourier transformation and an inverse discrete Fourier transformation combined with a translation model, and obtaining optimized 2D motion curves for cardiac movement, tremor movement, breathing movement and translational movement. The method is flexible and can be widely used in almost all angiography images (with a clear vascular distribution and covering two or more heart beat periods, which is easy to be satisfied) including double-armed X-ray angiography images.

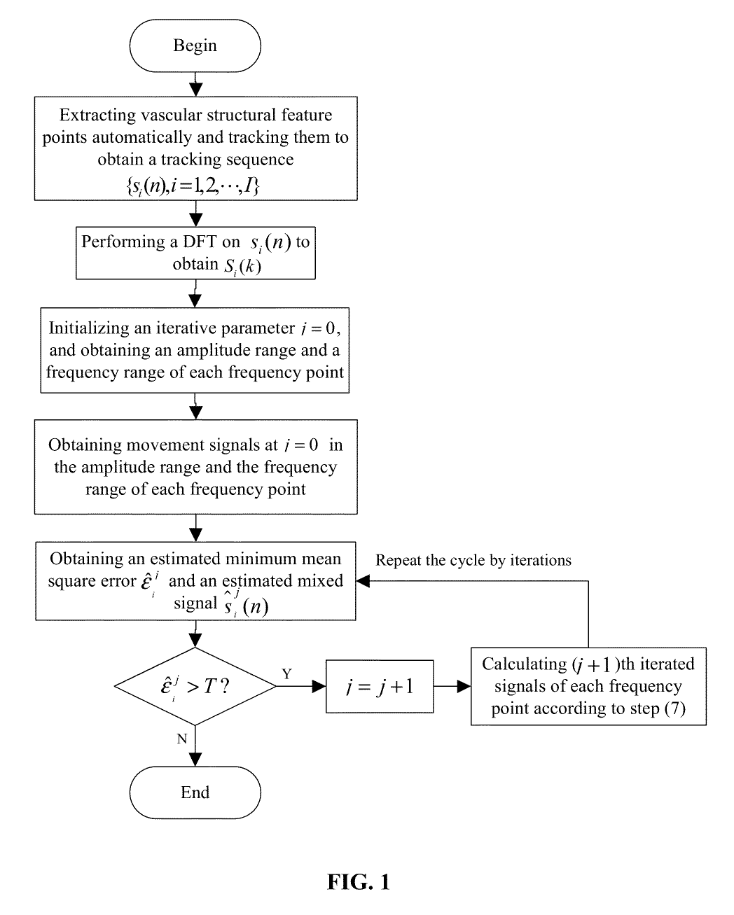

As in FIG. 1, a method for iteratively extracting motion parameters from angiography images using a multi-parameter model comprises steps of:

(1) extracting I vascular structural feature points automatically from a medical image of an angiography image sequence (where I is a natural number), and auto-tracking the feature points respectively in the angiography image sequence to obtain a tracking {sᵢ(n), i=1, . . . , I} sequence of each feature point, where n is frame number of the medical image in the angiography image sequence;

sᵢ(n) is expressed by the following equation:

sᵢ(n)=L(n)+rᵢ(n)+cᵢ(n)+hᵢ(n)+tᵢ(n),i∈[1, I],

where L(n) represents translational movement, rᵢ(n) represents breathing movement of an i^{th} feature point, cᵢ(n) represents cardiac movement of the i^{th} feature point, hᵢ(n) represents tremor movement of the i^{th} feature point, and tᵢ(n) represents other movements of the i^{th} feature point.

(2) performing a discrete Fourier transformation on the tracking sequence {sᵢ(n), i=1, . . . , I} of each feature point in (1) to obtain a discrete Fourier transformation result Sᵢ(k);

Sᵢ(k) is expressed by the following equation:

Sᵢ(k)=L(k)+Rᵢ(k)+Cᵢ(k)+Hᵢ(k),

where k represents a frequency point, and L(k), C(k), R(k) and H(k) represent harmonic coefficients of L(n), c(n) , r(n) and h(n) correspondingly and respectively.

(3) initializing an iterative parameter j=0, and obtaining an amplitude range and a frequency range of each frequency point of the discrete Fourier transformation result Sᵢ(k) in (2);

(4) performing a Fourier transformation on a tracking sequence of each frequency point in the amplitude range and the frequency range thereof to obtain Fourier transformation results L^{j}(k), Rᵢ^{j}(k), Cᵢ^{j}(k) and Hᵢ^{j}(k);

(5) performing an inverse Fourier transformation on the Fourier transformation results L^{j}(k), Rᵢ^{j}(k), Cᵢ^{j}(k) and Hᵢ^{j}(k) in (4), and obtaining an estimated minimum mean square error \hat{ε}ᵢ^{j} of each frequency point:

$${\hat{ɛ}}_{i}^{j} = min ( \frac{1}{N} \sqrt{{\sum }_{n} {( {s}_{i} ( n ) - {\hat{s}}_{i}^{j} ( n ) )}^{2}} ) ,$$

where ŝᵢ^{j}(n)=L^{j}(n)+rᵢ^{j}(n)+cᵢ^{j}(n)+hᵢ^{j}(n);

(6) determining whether the estimated minimum mean square error \hat{ε}ᵢ^{j} is greater than a predetermined threshold (a positive integer not greater than 2), and proceeding to (7) if yes, otherwise ending the process;

(7) processing spectrums L^{j}(k), Rᵢ^{j}(k) and Hᵢ^{j}(k) of each frequency point by a multi-parameter iterative optimizing algori^{th}m to obtain (j+1)^{th} iterated time-domain signals cᵢ^{j+1}(n), hᵢ^{j-1}(n), rᵢ^{j+1}(n), etc.;

Step (7) further comprises the following sub-steps of:

(7.1) calculating values L^{j}(k_{ic}), Rᵢ^{j}(k_{ic}) and Hᵢ^{j}(k_{ic}) of L^{j}(k), Rᵢ^{j}(k) and Hᵢ^{j}(k) near a frequency point k_{ic} in the frequency range respectively by the following equation while keeping L^{j}(k), Rᵢ^{j}(k) and Hᵢ^{j}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated cardiac signal spectrum by an equation Cᵢ^{j-1}(k_{ic})=Cᵢ⁰(k_{ic})−L^{j}(k_{ic})−Rᵢ^{j}(k_{ic})−Hᵢ^{j}(k_{ic}), performing a discrete Fourier transformation thereon in the frequency range, and obtaining an optimized (j+1)^{th} cardiac time-domain signal cᵢ^{j+1}(n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where ω is a frequency point after the discrete Fourier transformation, M represents a window size which is set to 3 normally, and Ŝᵢ^{j}(k)==L^{j}(k)+Rᵢ^{j+1}(k)+Cᵢ^{j+1}(k)+Hᵢ^{j}(k);

(7.2) calculating values L^{j}(kᵢₕ), Rᵢ^{j}(kᵢₕ) and Cᵢ^{j+1}(kᵢₕ) of L^{j}(k), Rᵢ^{j}(k) and Cᵢ^{j+1}(k) near a frequency point kᵢₕ in the frequency range respectively by the following equation while keeping L^{j}(k), Rᵢ^{j}(k) and Cᵢ^{j+1}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated high-frequency signal spectrum by an equation Hᵢ^{j+1}(kᵢₕ)=Hᵢ^{o}(kᵢₕ)−L^{j}(kᵢₕ)−Rᵢ^{j}(kᵢₕ)−Cᵢ^{j+1}(kᵢₕ), performing a discrete Fourier transformation thereon in the frequency range, and obtaining an optimized (j+1)^{th} high-frequency time-domain signal hᵢ^{j+1}(n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where Ŝᵢ^{j}(k)==L^{j}(k)+Rᵢ^{j}(k)+Cᵢ^{j+1}(k)+Hᵢ^{j-1}(k); and

(7.3) calculating values L^{j}(kᵢᵣ), Hᵢ^{j+1}(kᵢᵣ) and Cᵢ^{j+1}(kᵢᵣ) of L^{j}(k), Hᵢ^{j+1}(k) and Cᵢ^{j+1}(k) near a frequency point kᵢᵣ in the frequency range respectively by the following equation while keeping L^{j}(k), Hᵢ^{j+1}(k) and Cᵢ^{j+1}(k) constant:

$${X}_{p} ( k ) = {\sum }_{n = 0}^{N - 1} {x}_{p} ( n ) \cdot {e}^{- j ( \frac{2 π}{N} ) nk} ,$$

calculating a (j+1)^{th} iterated breathing signal spectrum by an equation Rᵢ^{j+1}(kᵢᵣ)=Rᵢ⁰(kᵢᵣ)−Iᵢ^{j}(kᵢᵣ)−Cᵢ^{j+1}(kᵢᵣ)−Hᵢ^{j+1}(kᵢᵣ), performing a discrete Fourier transformation thereon in the frequency range, and obtaining an optimized (j+1)t^{h} breathing time-domain signal rᵢ^{j-1}(n) by the following equation:

$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$

where Ŝᵢ^{j}(k)==L^{j}(k)+Rᵢ^{j+1}(k)+Cᵢ^{j+1}(k)+Hᵢ^{j+1}(k).

(8) processing a residual signal vᵢ^{j+1}(n)=sᵢ(n)−cᵢ^{j+1}(n)−hᵢ^{j+1}(n)−rᵢ^{j+1}(n) by a translation model to obtain a (j+1)^{th} iterated translation signal L^{j+1}(n);

(9) adding the (j+1)^{th} iterated time-domain signals cᵢ^{j+1}(n), hᵢ^{j+1}(n) and rᵢ^{j+1}(n) to the (j+1)^{th} iterated translation signal (n) to obtain an (j+1) iterated estimated mixed signal ŝᵢ^{j+1}(n), and calculating a (j+1)^{th} iterated minimum mean square error \hat{ε}ᵢ^{j+1}:

$${\hat{ɛ}}_{i}^{j + 1} = min ( \frac{1}{N} \sqrt{{\sum }_{n} {( {s}_{i} ( n ) - {\hat{s}}_{i}^{j + 1} ( n ) )}^{2}} ) ;$$

$$and$$

(10) determining whether the (j+1)^{th} iterated minimum mean square error \hat{ε}ᵢ^{j+1} is greater than the threshold in (6), and returning to (7) if yes, otherwise ending the process.

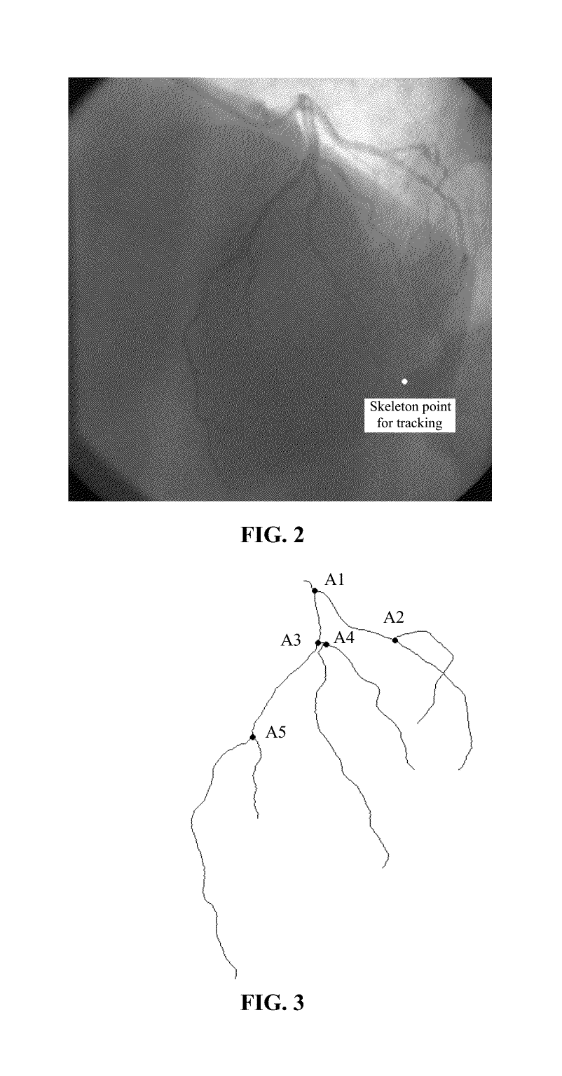

An angiography image of a patient 1 obtained from an angle of (50.8° , 30.2°) is shown in FIG. 2, and bifurcate points are extracted as structural feature points automatically, as shown in FIG. 3. A cardiac movement period equals 10 frames, and a sampling rate of an angiography image sequence is 12.5 frames per second. For angiography time is quite short, structural feature points A1-A4 with comparatively long residence times are selected for experiment.

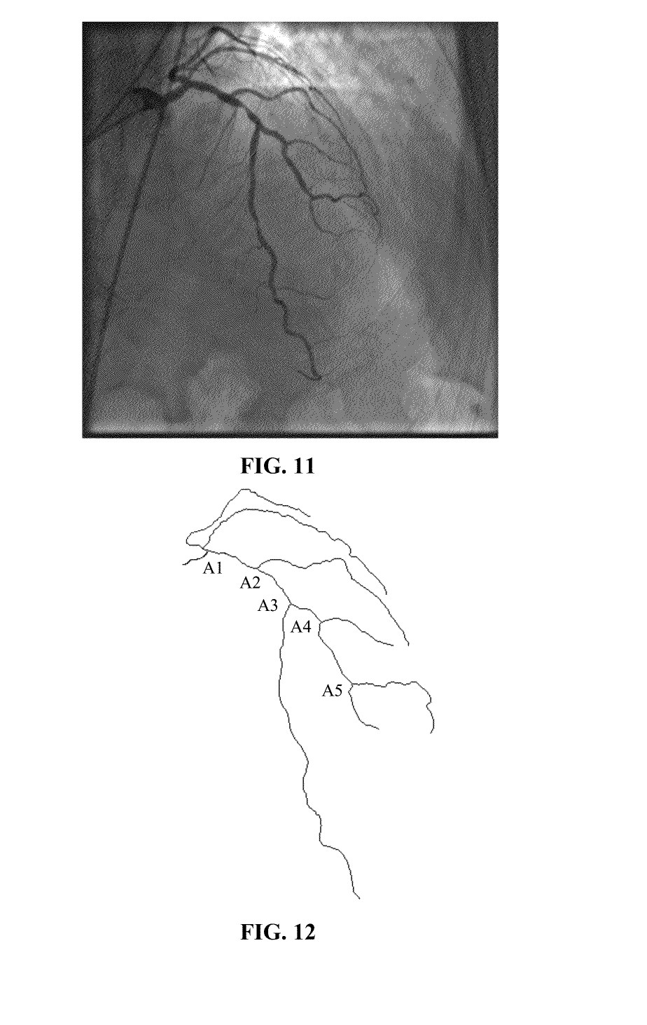

An angiography image of a patient 4 obtained from an angle of (30.8° , 15.3°) is shown in FIG. 11, and bifurcate points are extracted as structural feature points automatically, as shown in FIG. 12. The patient suffers from arrhythmia, two cardiac movement periods in a range of the patient's cardiac period are extracted by the method of the invention, equaling 9 frames and 12 frames respectively, and a sampling rate of an angiography image sequence is 12.5 frames per second.

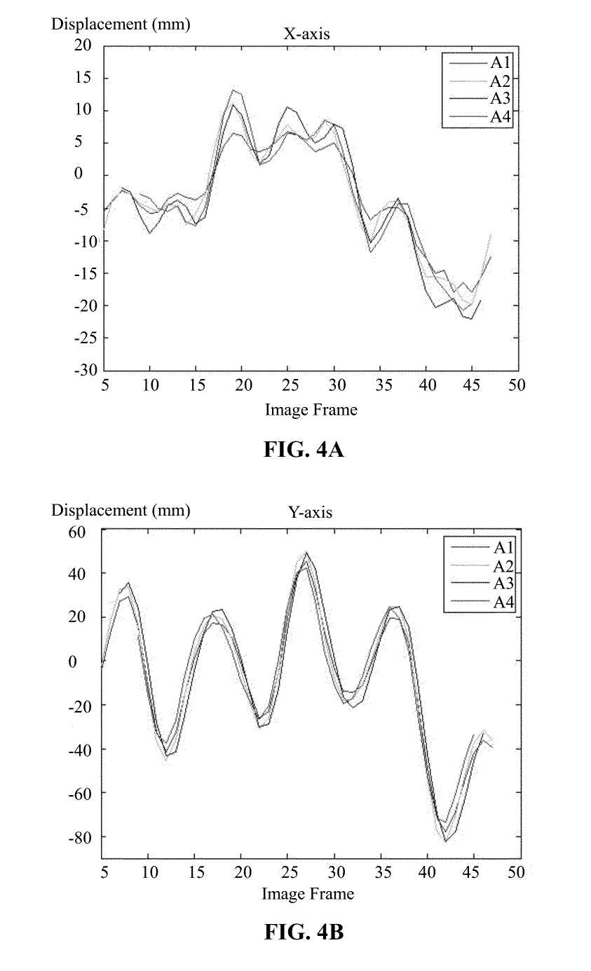

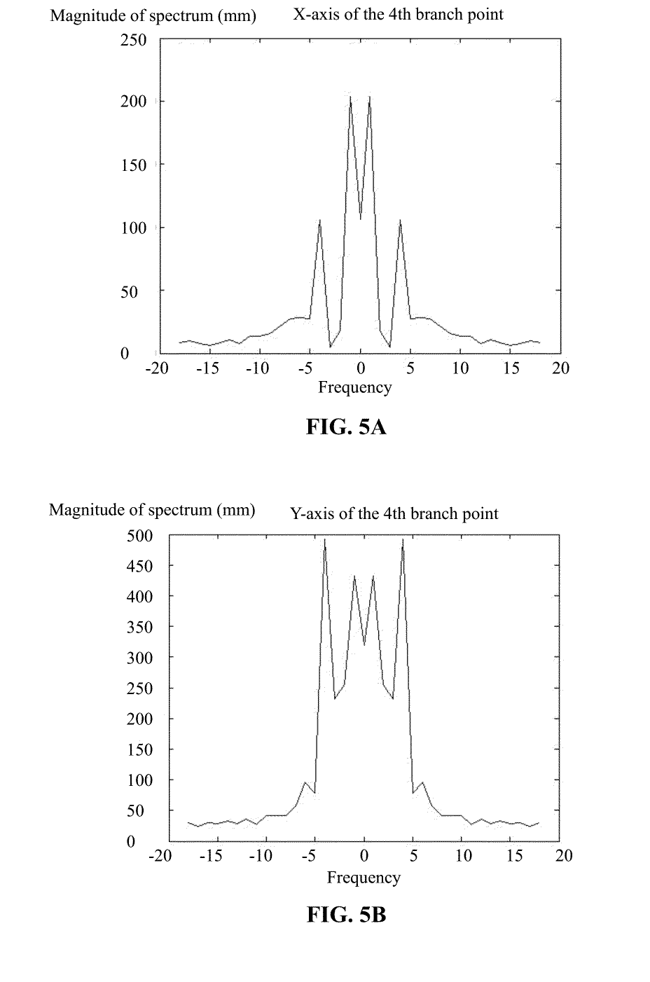

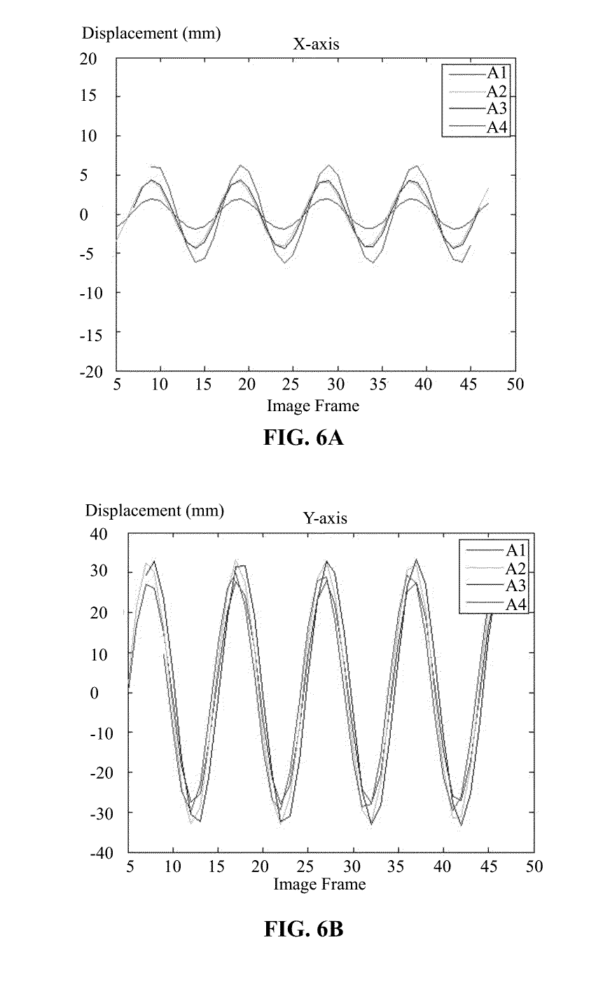

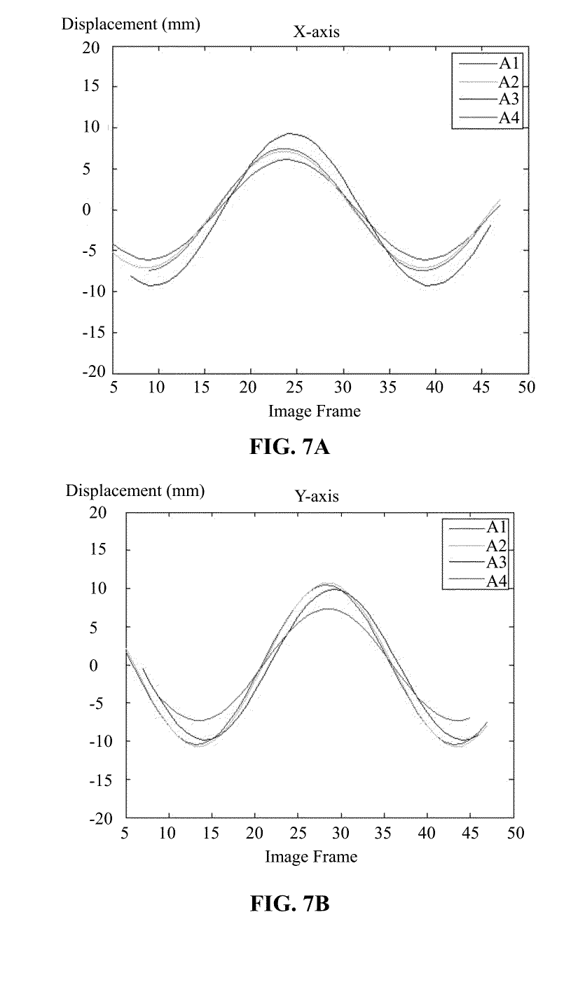

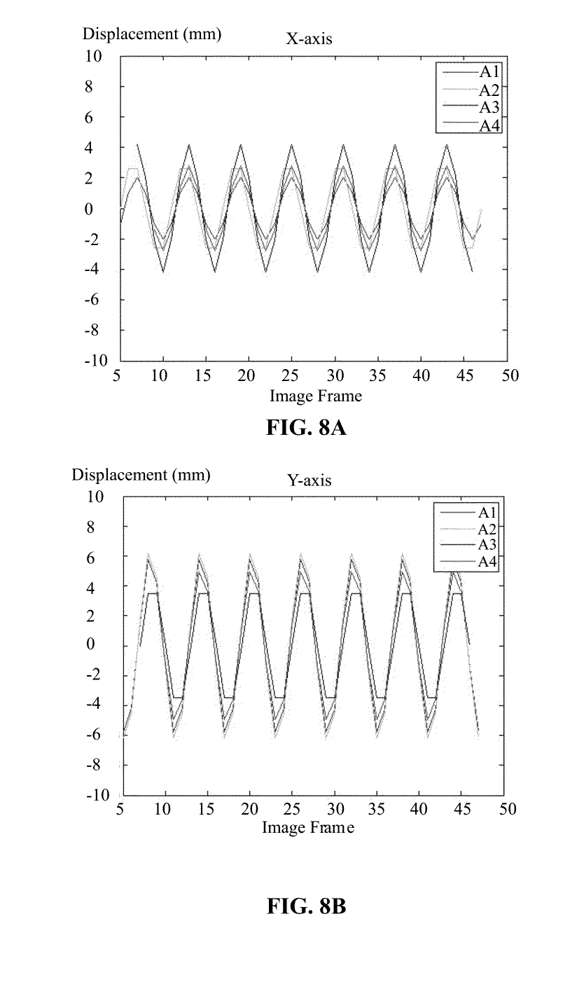

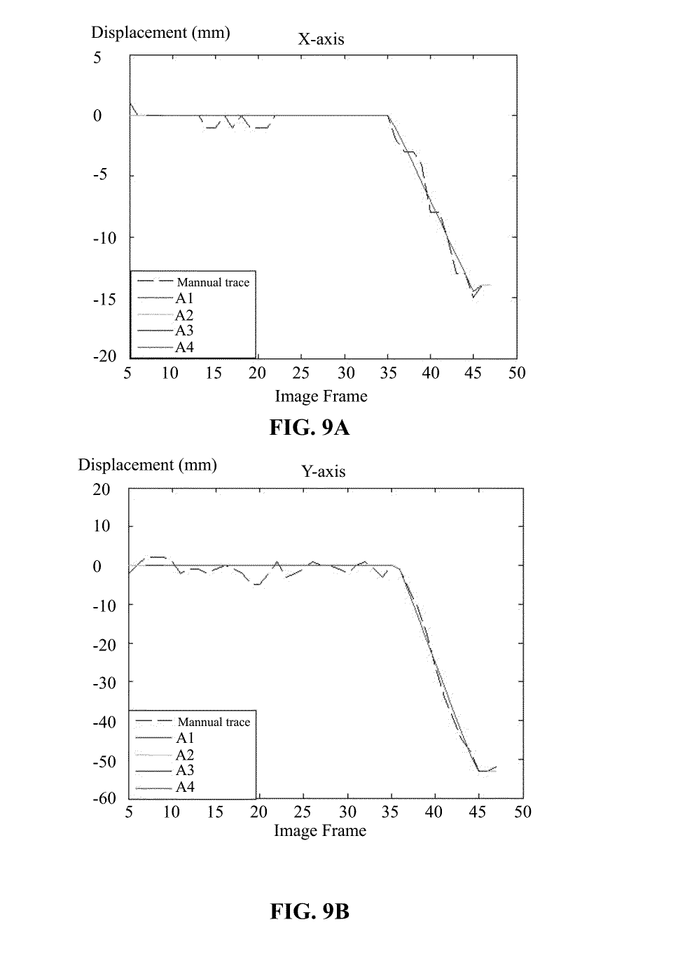

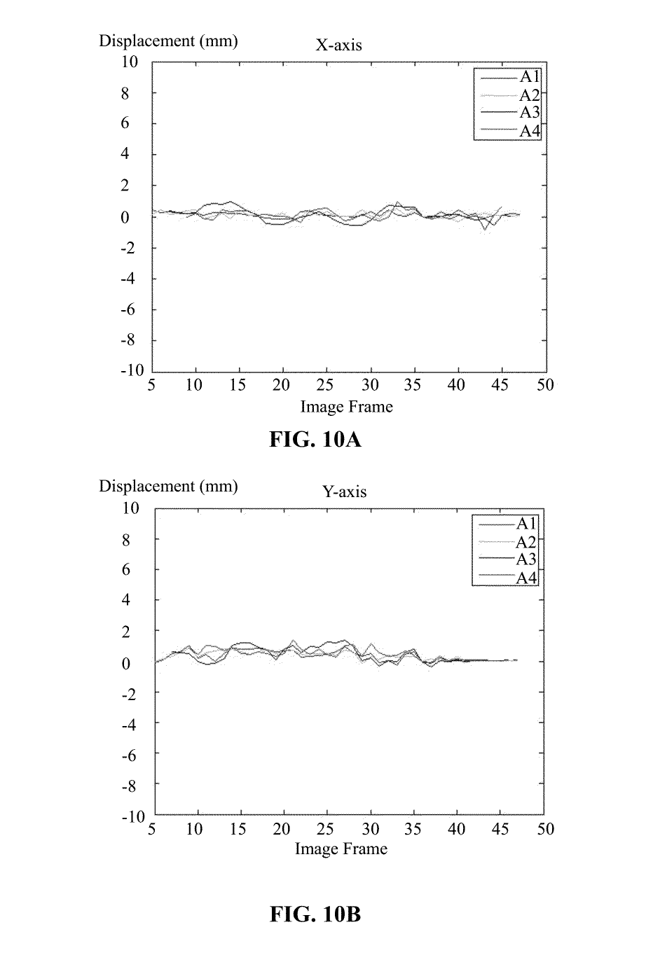

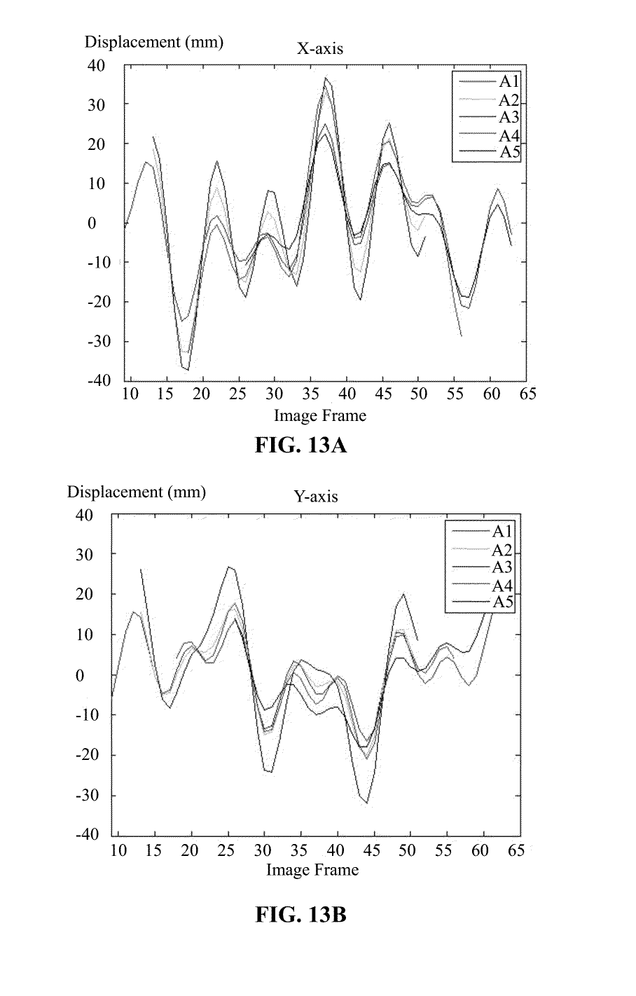

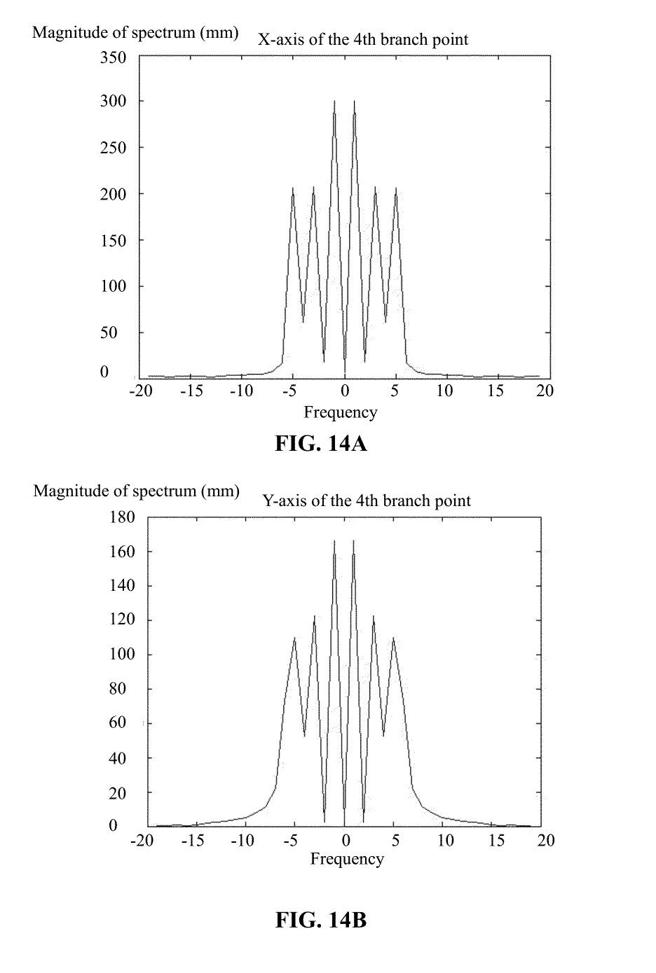

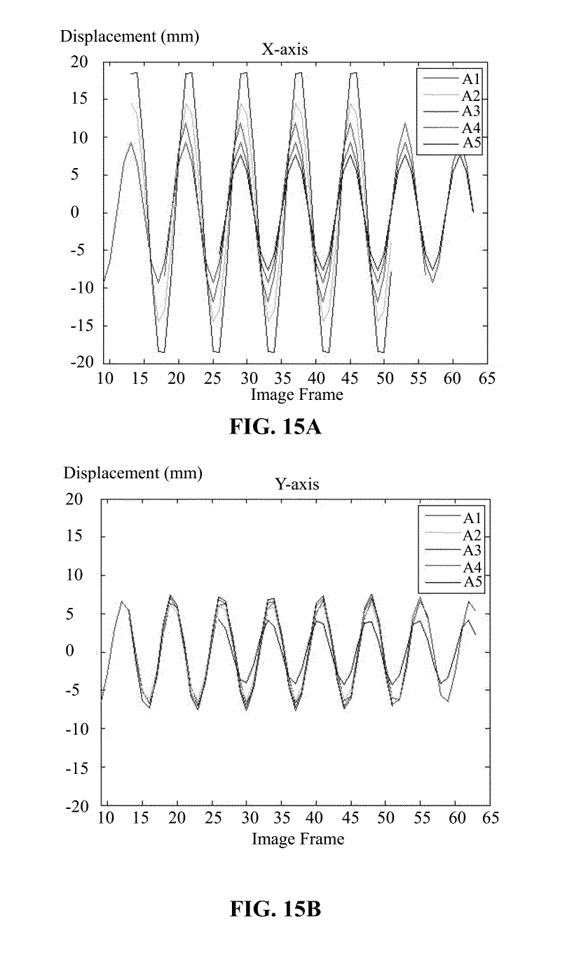

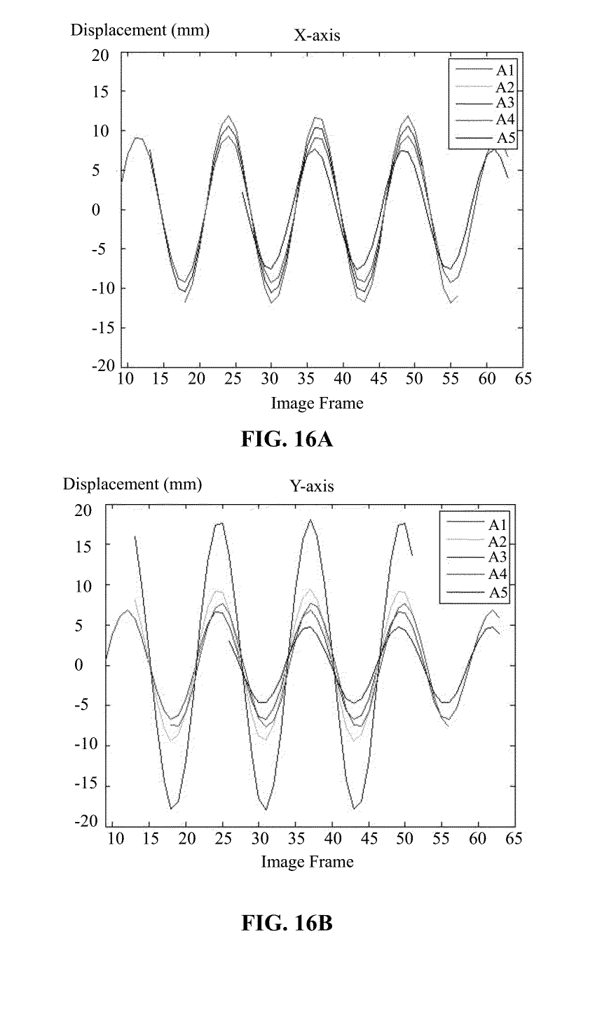

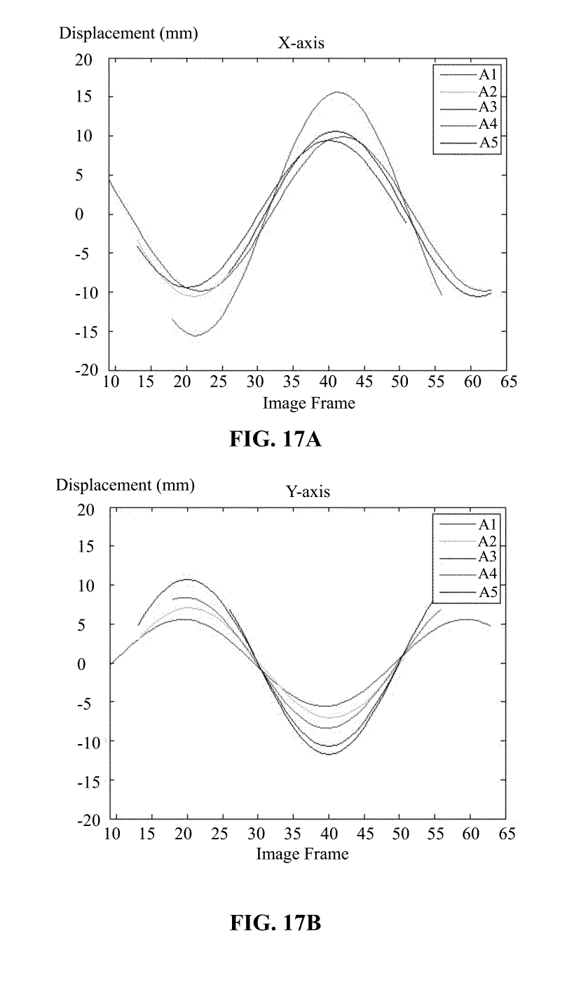

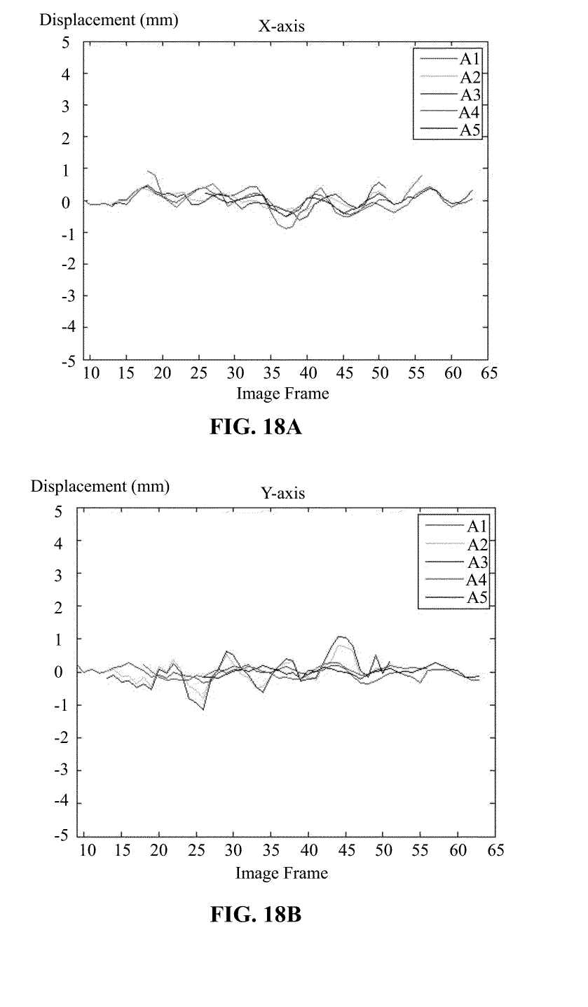

It can be seen from FIGS. 4A, 4B, 13A and 13B that original motion curves of the feature points are irregular and significantly influenced by breathing movement, and motion amplitudes are different for different feature points for the feature points are located on different parts of the heart with different movements. FIGS. 5A, 5B, 14A and 14B show spectrums obtained by performing a discrete Fourier transformation on mixed signals. It can be seen that each frequency point features an apparent peak, the number of which illustrates the number of frequency points contained in the mixed signal. In particular, as a peak appears at a frequency point 0, a translation signal must exist in the mixed signal. FIGS. 6A, 6B, 15A, 15B, 16A and 16B are extracted cardiac motion curves which show excellent periodicity. FIGS. 7A, 7B, 17A and 17B are extracted breathing motion curves which show excellent periodicity. It can be seen that peaks of the feature points occur almost simultaneously for angiography time is quite short and a corresponding phase change of each feature point is extremely small. Amplitudes of the peaks illustrate distribution of the feature points on a vascular surface, that is, a greater peak corresponds to a location closer to the lung. FIGS. 8A and 8B are extracted tremor signals which show that the patient trembled significantly during the treatment and amplitudes thereof are different with distribution of the feature points. FIGS. 9A and 9B illustrate extracted translational motion curves of feature points in FIG. 2 compared with those obtained by tracking skeleton points manually. It can be seen that results of the two methods are almost consistent in addition to errors caused by manual tracking. FIGS. 10A, 10B, 18A and 18B are residual curves of feature points in FIG. 11 after all movements being extracted. It can be seen that amplitudes thereof are smaller than 2 pixels which may be caused by segmentation and skeleton extraction, and motion components are extracted and separated completely.

Different from the patient 1 of FIG. 2, for the patient 4 of FIG. 11, there exist two different frequencies in a frequency range of a cardiac signal instead of a high frequency of tremor, which provides an important reference for diagnosis of a patient.

Overall, considering qualified feature points (such as intersections of ribs) do not exist in every angiography image in single-armed X-ray angiography, the invention extracts multiple motion components by selecting vascular structural feature points combined with filtering in frequency domain by Fourier transformations and optimizing multiple parameters, which is more flexible and can be widely used in almost all angiography images compared with obtaining breathing movement or translational movement merely by manual tracking. Besides, the method of the invention features a higher level of security and operability compared with setting labels on tissues adjacent to the heart and tracking them by related imaging methods, for the labels are usually intrusive and may damage a human body more or less, and the whole process of label setting, imaging and eliminating and breathing movement extraction is complicated which brings troubles and errors in operation inevitably.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for extracting motion parameters from angiography images, the method comprising:
(1) extracting I vascular structural feature points from a medical image of an angiography image sequence, and tracking the feature points respectively in the angiography image sequence to obtain a tracking sequence {sᵢ(n), i=1, . . . , I} of each feature point, where n is frame number of the medical image in the angiography image sequence;
(2) performing a discrete Fourier transformation on the tracking sequence {sᵢ(n), i=1, . . . , I} of each feature point in (1) to obtain a discrete Fourier transformation result Sᵢ(k);
(3) initializing an iterative parameter j=0, and obtaining an amplitude range and a frequency range of each frequency point of the discrete Fourier transformation result Sᵢ (k) in (2);
(4) performing a Fourier transformation on a tracking sequence of each frequency point in the amplitude range and the frequency range thereof to obtain Fourier transformation results;
(5) performing an inverse Fourier transformation on the Fourier transformation results in (4), and obtaining an estimated minimum mean square error of each frequency point;
(6) determining whether the estimated minimum mean square error is greater than a predetermined threshold, and proceeding to (7) if yes, otherwise ending the process;
(7) processing spectrums of each frequency point by a multi-parameter iterative optimizing algori^{th}m to obtain (j+1)^{th} iterated time-domain signals;
(8) processing a residual signal by a translation model to obtain a (j+1)^{th} iterated translation signal;
(9) adding the (j+1)^{th} iterated time-domain signals to the (j+1)^{th} iterated translation signal to obtain an (j+1)^{th} iterated estimated mixed signal, and calculating a (j+1)^{th} iterated minimum mean square error; and
(10) determining whether the (j+1)^{th} iterated minimum mean square error is greater than the threshold in (6), and returning to (7) if yes, otherwise ending the process.

2. The method of claim 1, wherein in (1), sᵢ(n) is expressed by the following equation:
sᵢ(n)=L(n)+rᵢ(n)+cᵢ(n)+hᵢ(n)+tᵢ(n),i∈[1, I],
where L(n) represents translational movement, rᵢ(n) represents breathing movement of an i^{th} feature point, cᵢ(n) represents cardiac movement of the i^{th} feature point, hᵢ(n) represents tremor movement of the i^{th} feature point, and tᵢ(n) represents other movements of the i^{th} feature point.

3. The method of claim 2, wherein in (2), ^{S}i (^{k}) is expressed by the following equation:
Sᵢ(k)=L(k)+Rᵢ(k)+Cᵢ(k)+Hᵢ(k),
where k represents a frequency point, and L(k), C(k), R(k) and H(k) represent harmonic coefficients of L(n), c(n), r(n) and h(n) correspondingly and respectively.

4. The method of claim 3, wherein in (5), the estimated minimum mean square error \hat{ε}ᵢ^{j} of the frequency point is expressed by the following equation:
$${\hat{ɛ}}_{i}^{j} = min ( \frac{1}{N} \sqrt{{\sum }_{n} {( {s}_{i} ( n ) - {\hat{s}}_{i}^{j} ( n ) )}^{2}} ) ,$$
where ŝᵢ^{j}(n)=L^{j}(n)+rᵢ^{j}(n)+cᵢ^{j}(n)+hᵢ^{j}(n).

5. The method of claim 4, wherein (7) further comprises the following sub-steps of:
(7.1) calculating values L^{j}(k_{ic}), Rᵢ^{j}(k_{ic}) and Hᵢ^{j}(k_{ic}IC) of L^{j}(k), Rᵢ^{j}(k) and Hᵢ^{j}(k) near a frequency point k_{ic} in the frequency range respectively by the following equation while keeping L^{j}(k), Rᵢ^{j}(k) and Hᵢ^{j}(k) constant:
$${X}_{p} ( k ) = {\sum }_{n = 0}^{N - 1} {x}_{p} ( n ) \cdot {e}^{- j ( \frac{2 π}{N} ) nk} ,$$
calculating a (j+1)^{th} iterated cardiac signal spectrum by an equation Cᵢ^{j+1}(k_{ic})=Cᵢ⁰(k_{ic})−Rᵢ^{j}(k_{ic})−Hᵢ^{j}(k_{ic}), performing a discrete Fourier transformation thereon in the frequency range, and obtaining an optimized (j+1)^{th} cardiac time-domain signal cᵢ^{j+1}(n) by the following equation:
$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$
where ω is a frequency point after the discrete Fourier transformation, M represents a window size which is set to 3 normally, and
Ŝᵢ^{j}(k)==L^{j}(k)+Rᵢ^{j}(k)+Cᵢ^{j+1}(k)+Hᵢ^{j}(k);
(7.2) calculating values L^{j}(kᵢₕ), Rᵢ^{j}(kᵢₕ) and Cᵢ^{j+1}(kᵢₕ) of L^{j}(k), Rᵢ^{j}(k) and Cᵢ^{j+1}(k) near a frequency point kᵢₕ in the frequency range respectively by the following equation while keeping L^{j}(k), Rᵢ^{j}(k) and Cᵢ^{j+1}(k) constant:
$${X}_{p} ( k ) = {\sum }_{n = 0}^{N - 1} {x}_{p} ( n ) \cdot {e}^{- j ( \frac{2 π}{N} ) nk} ,$$
calculating a (j+1)^{th} iterated high-frequency signal spectrum by an equation Hᵢ^{j+1}(kᵢₕ)=Hᵢ⁰(kᵢₕ)−L^{j}(kᵢₕ)−Rᵢ^{j}(kᵢₕ)−Cᵢ^{j+1}(kᵢₕ), performing a discrete Fourier transformation thereon in the frequency range, and obtaining an optimized (j+1)^{th} high-frequency time-domain signal hᵢ^{j-1}(n) by the following equation:
$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$
where Ŝᵢ^{j}(k)==L^{j}(k)+Rᵢ^{j}(k)+Cᵢ^{j+1}(k)+Hᵢ^{j-1}(k); and
(7.3) calculating values L^{j}(kᵢᵣ), Hᵢ^{j+1}(kᵢᵣ) of L^{j}(k), Hᵢ^{j+1}(k) and Cᵢ^{j+1}(k) near a frequency point kᵢᵣ in the frequency range respectively by the following equation while keeping L^{j}(k), Hᵢ^{j+1}(k) and Cᵢ^{j+1}(k) constant:
$${X}_{p} ( k ) = {\sum }_{n = 0}^{N - 1} {x}_{p} ( n ) \cdot {e}^{- j ( \frac{2 π}{N} ) nk} ,$$
calculating a (j+1)^{th} iterated breathing signal spectrum by an equation Rᵢ^{j+1}(kᵢᵣ)=Rᵢ⁰(kᵢᵣ)−L^{j}(kᵢᵣ)−Cᵢ^{j-1}(kᵢᵣ)−Hᵢ^{j+1}(kᵢᵣ), performing a discrete Fourier transformation thereon in the frequency range, and obtaining an optimized (j+1)^{th} breathing time-domain signal rᵢ^{j+1}(n) by the following equation:
$${\hat{e}}_{i}^{j} = min ( {\sum }_{k = ω - M}^{ω + M} {( {S}_{i} ( k ) - {\hat{S}}_{i}^{j} ( k ) )}^{2} ) ,$$
where Ŝᵢ^{j}(k)==L^{j}(k)+Rᵢ^{j+1}(k)+Cᵢ^{j+1}(k)+Hᵢ^{j+1}(k).

6. The method of claim 5, wherein in (9), the (j+1)^{th} iterated minimum mean square error \hat{ε}ᵢ^{j+1} is expressed by the following equation:
$${\hat{ɛ}}_{i}^{j + 1} = min ( \frac{1}{N} \sqrt{{\sum }_{n} {( {s}_{i} ( n ) - {\hat{s}}_{i}^{j + 1} ( n ) )}^{2}} ) .$$

7. A system for extracting motion parameters from angiography images, the system comprising:
a) a first module, operable for extracting I vascular structural feature points from a medical image of an angiography image sequence, and tracking the feature points respectively in the angiography image sequence to obtain a tracking sequence {sᵢ(n), i=1, . . . , I} of each feature point, where n is frame number of the medical image in the angiography image sequence;
b) a second module, operable for performing a discrete Fourier transformation on the tracking sequence {sᵢ(n), i=1, . . . , I} of each feature point derived by the first module to obtain a discrete Fourier transformation result Sᵢ(k);
c) a third module, operable for initializing an iterative parameter j=0, and obtaining amplitude range and frequency range of each frequency point of the discrete Fourier transformation result Sᵢ(k) derived by the second module;
d) a fourth module, operable for performing a Fourier transformation on a tracking sequence of each frequency point in the amplitude range and the frequency range thereof to obtain Fourier transformation results;
e) a fifth module, operable for performing an inverse Fourier transformation on the Fourier transformation results derived by the fourth module, and obtaining an estimated minimum mean square error of each frequency point;
f) a sixth module, operable for determining whether the estimated minimum mean square error is greater than a predetermined threshold, and proceeding to a seventh module if yes, otherwise ending the process;
g) a seventh module, operable for processing spectrums of each frequency point by a multi-parameter iterative optimizing algori^{th}m to obtain (j+1)^{th} iterated time-domain signals;
h) an eighth module, operable for processing a residual signal by a translation model to obtain a (j+1)^{th} iterated translation signal;
i) a ninth module, operable for adding the (j+1)^{th} iterated time-domain signals to the (j+1)^{th} iterated translation signal to obtain an (j+1)^{th} iterated estimated mixed signal, and calculating a (j+1)^{th} iterated minimum mean square error; and
j) a tenth module, operable for determining whether the (j+1)^{th} iterated minimum mean square error is greater than the threshold in the sixth module, and returning to the seventh module if yes, otherwise ending the process.
